(19) United States
(12) Patent Application Publication
(10) Pub. No.: US 2026/0014303 A1
(43) Pub. Date: Jan. 15, 2026

(54) SORBENT CARTRIDGE FOR USE IN A DIALYSIS SYSTEM

(71) Applicant: Mozarc Medical US LLC, Minneapolis, MN (US)

(72) Inventors: Sukalyan Dutta, Minneapolis, MN (US); Peter Orson, Minneapolis, MN (US)

(21) Appl. No.: 19/265,141

(22) Filed: Jul. 10, 2025

Related U.S. Application Data
(60) Provisional application No. 63/669,867, filed on Jul. 11, 2024.

(51) Int. Cl. A61M 1/14

(52) U.S. Cl. CPC A61M 1/1562; A61M 1/1566

(57) ABSTRACT

A sorbent cartridge for filtering dialysate in a dialysis system is disclosed. The cartridge can include a fluid inlet, a fluid outlet, a plurality of sorbent materials arranged in series between the fluid inlet and the fluid outlet, and a recharging solution disposed in a container. Each of the plurality of sorbent materials can be disposed in a respective replaceable container. The plurality of sorbent materials can be arranged for fluid flow in a longitudinal direction of the respective replaceable container. The recharging solution can be selectively fluidly connected to one or more of the plurality of sorbent materials.

### CROSS REFERENCE TO RELATED APPLICATIONS

The present application claims priority to and the benefit of U.S. Provisional Patent Application 63/669,867, filed Jul. 11, 2024, the contents of which is hereby incorporated by reference in its entirety.

### FIELD

This disclosure relates generally to a sorbent cartridge. More particularly, this disclosure relates to a sorbent cartridge configured for use in a dialysis system.

### BACKGROUND

Dialysis systems can be used to treat patients with kidney disorders. There are a number of dialysis systems in use in the health care industry. Sorbent cartridges are used in these dialysis systems for treatment of the patients to remove the waste products from the blood.

### SUMMARY

In some embodiments, a sorbent cartridge includes a fluid inlet; a fluid outlet; and a plurality of sorbent materials arranged in series between the fluid inlet and the fluid outlet. In some embodiments, each of the plurality of sorbent materials is configured to be disposed in a respective replaceable container. In some embodiments, the plurality of sorbent materials are configured for fluid flow in a longitudinal direction of the respective replaceable container. In some embodiments, a recharging solution is disposed in a container. In some embodiments, the recharging solution is configured to be selectively fluidly connected to one or more of the plurality of sorbent materials.

In some embodiments, the respective replaceable container has a length and a diameter. In some embodiments, the respective replaceable container has an aspect ratio defined by a ratio of the length to the diameter (length:diameter). In some embodiments, the aspect ratio is at least 1.5. In some embodiments, the aspect ratio is at least 3.

In some embodiments, one or more of the plurality of sorbent materials is in a form of a sorbent block.

In some embodiments, the plurality of sorbent materials include one or more of activated carbon, zirconium phosphate, or zirconium oxide.

In some embodiments, the sorbent cartridge has a cylindrical geometry. In some embodiments, the plurality of sorbent materials are arranged circumferentially around the sorbent cartridge in an order of flow.

In some embodiments, a dissolvable element is included within one or more of the plurality of sorbent materials.

In some embodiments, the dissolvable element is configured to prevent particle segregation of a respective sorbent material in the respective replaceable container.

In some embodiments, the respective replaceable container is configured to be installed within the sorbent cartridge using a snap in loader.

In some embodiments, the plurality of sorbent materials are configured for fluid flow in a second direction other than the longitudinal direction.

In some embodiments, the second direction includes a radial direction, a circumferential direction, or combination thereof.

In some embodiments, a dialysis system is configured to output a fresh dialysate to a patient and configured to receive a used dialysate from the patient. In some embodiments, the dialysis system includes a sorbent cartridge configured to receive the used dialysate and filter to generate a refreshed dialysate. In some embodiments, the sorbent cartridge includes a fluid inlet configured to receive the used dialysate; a fluid outlet configured to output the refreshed dialysate; and a plurality of sorbent materials arranged in series between the fluid inlet and the fluid outlet. In some embodiments, each of the plurality of sorbent materials is configured to be disposed in a respective replaceable container. In some embodiments, the plurality of sorbent materials are configured for fluid flow in a longitudinal direction of the respective replaceable container. In some embodiments, a recharging solution is disposed in a container, the recharging solution configured to be selectively fluidly connected to one or more of the plurality of sorbent materials.

In some embodiments, the respective replaceable container has a length and a diameter. In some embodiments, the respective replaceable container has an aspect ratio defined by a ratio of the length to the diameter (length:diameter). In some embodiments, the aspect ratio is at least 1.5. In some embodiments, the aspect ratio is at least 3.

In some embodiments, one or more of the plurality of sorbent materials are in a form of a sorbent block.

In some embodiments, the plurality of sorbent materials include one or more of activated carbon, zirconium phosphate, or zirconium oxide.

In some embodiments, the sorbent cartridge has a cylindrical geometry. In some embodiments, the plurality of sorbent materials are arranged circumferentially around the sorbent cartridge in an order of flow.

In some embodiments, a dissolvable element is disposed within one or more of the plurality of sorbent materials.

In some embodiments, the dissolvable element is configured to prevent particle segregation of a respective sorbent material in the respective replaceable container.

In some embodiments, the respective replaceable container is configured to be installed within the sorbent cartridge using a snap in loader.

In some embodiments, the plurality of sorbent materials are configured for fluid flow in a second direction other than the longitudinal direction.

In some embodiments, the second direction includes a radial direction, a circumferential direction, or combination thereof.

In some embodiments, a device includes a housing including a fluid inlet and a fluid outlet; and a plurality of terminals configured to be fluidly connected to a connector of a respective sorbent material. In some embodiments, the plurality of terminals are fluidly connected so that the respective sorbent material is fluidly connected when the respective sorbent material is installed in the plurality of terminals. In some embodiments, the device includes a recharging solution container configured to be selectively fluidly connected to one or more of the plurality of terminals.

In some embodiments, the housing is cylindrical and the plurality of terminals are arranged about a circumference of the housing.

In some embodiments, the respective sorbent material includes one or more of activated carbon, zirconium phosphate, or zirconium oxide.

In some embodiments, a first of the plurality of terminals is configured to receive the zirconium phosphate or the zirconium oxide. In some embodiments, the recharging solution container is configured to be selectively fluidly connected to the first of the plurality of terminals.

### BRIEF DESCRIPTION OF THE DRAWINGS

References are made to the accompanying drawings that form a part of this disclosure and that illustrate embodiments in which the systems and methods described in this Specification can be practiced.

FIG. 1 is a schematic diagram of a dialysis system, according to some embodiments.

FIG. 2 is a perspective view of a replaceable container for a sorbent cartridge, according to some embodiments.

FIG. 3 is a schematic diagram of a sorbent cartridge, according to some embodiments.

FIG. 4 is a schematic diagram of a sorbent cartridge, according to some embodiments.

Like reference numbers represent the same or similar parts throughout.

### DETAILED DESCRIPTION

Dialysis systems such as, but not limited to, hemodialysis, hemofiltration, hemodiafiltration, and peritoneal dialysis, can utilize a sorbent cartridge to filter waste products from the blood. The sorbent cartridges disclosed are designed to increase the efficiency of filtering materials and reduce patient cost for in-home dialysis systems.

Dialysis is the most commonly applied physical principle to address the build-up of urea and other toxins, to balance electrolytes, and to remove excess fluid in patients with kidney failure. Dialysis as a renal or kidney replacement therapy can include hemodialysis, hemodiafiltration, or hemofiltration to remove toxins and impurities from a patient's blood. Dialysis membranes employed in dialysis treatment are typically only selective toward molecular weight and not toward other properties such as electrical charge. As such, urea, ions and other small molecules can move across the dialysis membrane unimpeded from a higher concentration to a lower concentration via diffusion thereby lowering the concentration of such species in the patient's blood through the process of hemodialysis.

In general, dialysis systems can rely upon the regeneration of used dialysate (i.e., dialysate having urea and/or other waste species therein) to form refreshed dialysate that can be reused to perform dialysis or to dialyze the patient utilizing a smaller dialysate volume. Regeneration of the dialysate allows for the volume of fluid that needs to be supplied to perform a session of dialysis treatment to be limited and enable a portable system. In the present invention, a dialysis circuit is provided for conveying and re-circulating a dialysate between a dialyzer, where the dialysate picks up impurities such as urea, and a sorbent cartridge where waste species are removed from the dialysate to form refreshed dialysate after the addition of cation electrolytes.

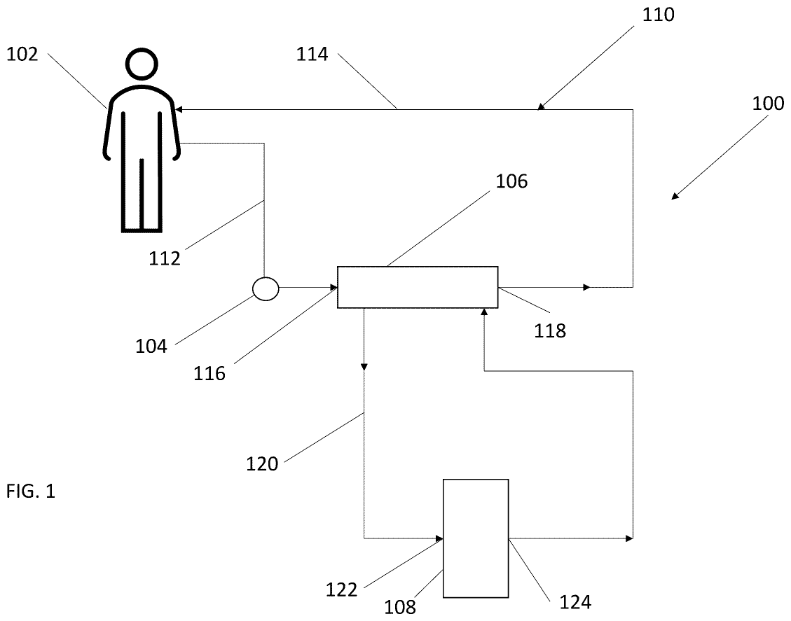

FIG. 1 is a schematic diagram of a dialysis system 100, according to some embodiments. FIG. 1 shows the dialysis system 100 for circulating blood and a dialysate through a dialyzer 106. The dialysis system 100 can be configured to output a fresh dialysate to a patient and to receive a used dialysate from the patient. In some embodiments, the “used dialysate” can alternatively be referred to as a “spent dialysate” or the like. A shunt such as a needle or catheter is connected to a patient's 102 vasculature to draw blood and circulate the patient's 102 blood through an extracorporeal circuit 110. The portion of the extracorporeal circuit 110 that contains drawn blood from the patient can be referred to as the arterial line 112, which by convention is understood to mean a line for transporting blood from the patient regardless of whether blood is drawn from an artery or vein of the patient 102. Similarly, the portion that returns blood to the patient 102 can be referred to as the venous line 114. The arterial line 112 and the venous line 114 can connect with one or more veins of the patient 102. The blood can be moved through the extracorporeal circuit 110 by a blood pump 104, which is typically located along the arterial line 112. The blood pump 104 can convey blood through the dialyzer 106 where the blood is contacted with a blood side of a high permeability dialysis membrane. Blood enters the dialyzer 106 through a blood inlet 116 and exits through a blood outlet 118. On the other side of the dialysis membrane within the dialyzer 106 is a dialysate solution.

Dialysate within the system can be conveyed through a dialysate pathway 120, which can carry dialysate to the dialyzer 106. Dialysate that can be conveyed through the dialyzer 106 picks up waste products from the blood, including urea, by diffusion, hemofiltration, or hemodiafiltration. The dialysate travels through the dialysate pathway 120 and is passed through a sorbent cartridge 108 to remove the waste products before being re-conveyed through the dialyzer 106. As used herein, a “sorbent cartridge” refers to a cartridge containing one or more sorbent materials for removing specific solutes (such as, but not limited to, urea or the like) from solution. The dialysate can enter the sorbent cartridge 108 at a dialysate fluid inlet 122 and can exit at a dialysate fluid outlet 124. More specifically, the used dialysate, containing waste products taken from the blood in the dialyzer 106, enters the sorbent cartridge 108 at dialysate fluid inlet 122. The dialysate can then be filtered through the sorbent cartridge 108. Once filtered, the dialysate will exit the sorbent cartridge 108 at the dialysate fluid outlet 124 as a refreshed dialysate. The refreshed dialysate can then be re-introduced to the dialyzer 106 to remove more waste products from the blood contained therein. By re-filtering or recycling the dialysate, the total volume of dialysate required for a dialysis therapy can be reduced, thereby reducing a size of a dialysate reservoir.

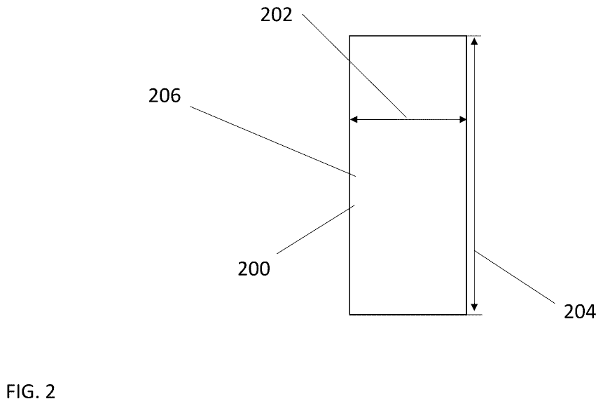

Turning to FIG. 2, a plurality of replaceable containers 200 for the sorbent cartridge 108 can be configured to receive the used dialysate. Each of the plurality of replaceable containers 200 for sorbent cartridge 108 can be a cylinder or have cylindrical geometry. A cylindrical geometry is merely an example and the actual geometry of the sorbent cartridge 108 can vary according to the principles of this disclosure. In known sorbent cartridges, an aspect ratio defined by the ratio of length to diameter (e.g., length:diameter or L/D) of 1 is typical. However, the sorbent cartridge 108 disclosed herein can have an aspect ratio defined by a ratio of a length 204 to a diameter 202 (e.g., length:diameter or L/D) of at least 1.5, or about 1.5 to about 4.5, for example about 2 to about 4, or from about 2.5 to about 3.5, or at least 3. In other words, the diameters of the plurality of replaceable containers 200 can be minimized while the lengths can be extended. In some embodiments, the aspect ratio of each of the plurality of replaceable containers 200 may have about 2× to about 8× aspect ratio compared to the aspect ratio of prior sorbent cartridges, where ‘X’ is the aspect ratio of prior sorbent cartridges. This can have the effect of miniaturizing the size of the plurality of replaceable containers 200 and the overall sorbent cartridge 108. As the system 100 can be designed for at-home dialysis use, minimizing the size of the system 100 can be beneficial to patients and end users. The larger aspect ratio can increase the effectiveness of the filtering materials within each of the plurality of replaceable containers 200. For example, by increasing the efficiency of ion exchange within the plurality of replaceable containers 200. As the efficiency of each of the plurality of replaceable containers 200 is increased, less filtering material can be used in each of the plurality of replaceable containers 200, thereby reducing cost for the patient 102. Because of the high aspect ratio of the plurality of replaceable containers 200, blocks of the sorbent materials may be used, rather than traditional powder of the sorbent materials. For example, blocks of activated carbon can be used rather than powdered activated carbon. However, powdered activated carbon can still be used in the system 100. Being able to use blocks of the sorbent materials can lead to more predictable filtration and less variability than using powdered sorbent materials. In some embodiments, including the sorbent materials in a block form instead of a powdered form can provide for a more structured arrangement of the sorbent material. As a result, a pressure drop through the sorbent material can be reduced relative to a sorbent material in a powdered form.

The plurality of replaceable containers 200 can have a diameter 202 of about 1 inch to about 3 inches. As used herein, “about” can include variations subject to, for example, manufacturing tolerances or the like. Additionally, the plurality of replaceable containers 200 can have a length 204 of about 5 inches to about 10 inches. The plurality of replaceable containers 200 can be disposed in a housing to form the sorbent cartridge 108. In some embodiments, the housing can be a manifold and thus only house or contain a part of the plurality of replaceable containers 200. It is to be appreciated that these lengths and diameters are examples and that the actual dimensions of the plurality of replaceable containers 200 can vary according to the principles of this disclosure.

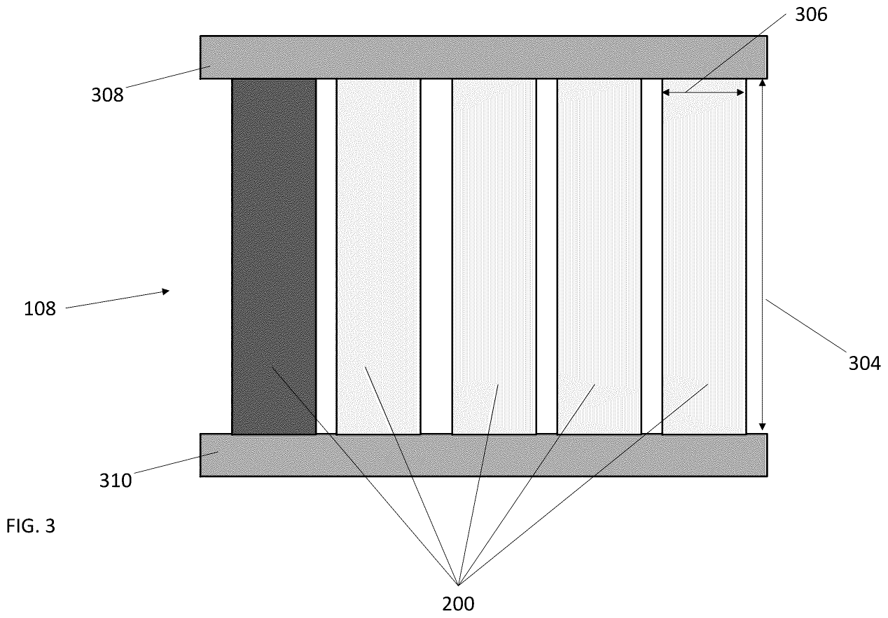

Referring to FIG. 3, the plurality of replaceable containers 200 forming the sorbent cartridge 108, through which the dialysate is conveyed to remove various waste products, can be arranged in the plurality of replaceable containers 200, fluid bed reactors, or terminals, to form the sorbent cartridge 108. For example, the plurality of replaceable containers 200 can be located along the circumference of the sorbent cartridge 108 and each of the plurality of replaceable containers 200 can contain a material to filter the dialysate or to process the dialysate. The plurality of replaceable containers 200 can be configured to extend along the length 204 of the sorbent cartridge 108. For example, where the sorbent cartridge 108 is a cylinder, the plurality of replaceable containers 200 can extend from one end to an opposing end. In other words, the plurality of sorbent materials within the plurality of replaceable containers 200 can be configured for fluid flow in a longitudinal direction of the respective one of the plurality of replaceable containers 200. In some embodiments, the fluid flow in the longitudinal direction can alternatively be referred to as the axial direction. It is to be appreciated that in addition to flow in the longitudinal direction, there can be flow in one or more other directions such as the radial direction, the circumferential direction, tangential, combinations thereof, or the like.

While shown as cylinders, the plurality of replaceable containers 200 can be any shape and may vary according to the principles of this disclosure. For example, the plurality of replaceable containers 200 can be squared, ‘pie slice shaped,’ or any conceivable polygonal shape configured to fit within the sorbent cartridge 108. The plurality of replaceable containers 200 can be configured to be installed within the sorbent cartridge 108 using a snap in loader. It is to be appreciated that this is one type of installation and that others are possible within the scope of this disclosure. For example, the plurality of replaceable containers 200 can be installed using sliding (e.g., drop in), a threaded connection, or the like.

Before installation, the plurality of replaceable containers 200 can be in a closed configuration, defined by a closed flow path. While in the closed configuration, no particulate, dust, or other contaminants can enter into the plurality of replaceable containers 200. The plurality of replaceable containers 200 can be configured to switch to an open configuration when installed into the sorbent cartridge 108, thereby opening the flow path and allowing dialysate to flow through the respective one of the plurality of replaceable containers 200 as installed. The switch from closed to open configurations can happen manually, by the user, or as a direct result of being installed, or as a direct result of the snap in or other connection type. It is to be appreciated that this is one type of installation and that others are possible within the scope of this disclosure. In some embodiments, the sorbent cartridge 108 and the plurality of replaceable containers 200 can include one or more additional features used for confirming that the correct sorbent materials are installed. For example, the plurality of replaceable containers 200 can include an electronic device such as, but not limited to, a radio frequency identification (RFID) device that the dialysis system 100 can use to confirm the correct sorbent materials are installed in the sorbent cartridge 108. In some embodiments, the sorbent cartridge 108 can also include an RFID device so that the dialysis system 100 can confirm the correct sorbent cartridge 108 is installed within the dialysis system 100.

The plurality of replaceable containers 200 can be designed to be modular, permitting case of removal or installation of the plurality of replaceable containers 200 into the sorbent cartridge 108. Through the case of installation, a user (e.g., the patient 102, a medical technician, a clinician, or the like) can add, or remove, to the plurality of replaceable containers 200 based on the mass of the patient 102, length of the therapy necessary, or doctor recommendation. For example, if the patient 102 has a larger mass than typical, more of the plurality of replaceable containers 200 can be added into the sorbent cartridge 108 for their dialysis treatment, thereby reducing the need to manufacture a customized sorbent cartridge 108 tailored to the needs of each patient 102. In some embodiments, a limitation on the number of plurality of replaceable containers 200 can be limited to a total number of fluid connections. It is to be appreciated that the exact number of plurality of replaceable containers 200 can vary according to the principles of this disclosure. Alternatively, if the patient 102 has a larger mass than typical, a replaceable container with more filtering material than normal may be added into the sorbent cartridge 108, thereby reducing the need to manufacture a customized sorbent cartridge 108 tailored to the needs of each patient 102. The modular design can save money on plastic manufacturing and associated costs by preventing the need for large, custom-made polypropylene cartridges by allowing the patient 102 to add or remove the necessary ones of the plurality of replaceable containers 200 for their specific dialysis treatment. By removing the need for custom made cartridges, the modular design can decrease the cost required to provide quality dialysis treatment tailored to the specific needs of each patient 102.

As illustrated in FIG. 3, the sorbent cartridge 108 can include a first manifold 308 and a second manifold 310 via which the plurality of replaceable containers 200 are fluidly connected. In some embodiments, the sorbent cartridge 108 can include a housing extending between the first manifold 308 and the second manifold 310 to house the plurality of replaceable containers 200. In some embodiments, the first manifold 308 can be an inlet side manifold configured to include the dialysate fluid inlet 122 and the second manifold 310 can be an outlet side manifold configured to include the dialysate fluid outlet 124. It is to be appreciated that these configurations can be reversed. In some embodiments, either the first manifold 308 or the second manifold 310 can be present and can include both the dialysate fluid inlet 122 and the dialysate fluid outlet 124. The first manifold 308 and the second manifold 310 can be configured to distribute fluid among the plurality of replaceable containers 200 forming the sorbent cartridge 108. In some embodiments, a first replaceable container of the plurality of replaceable containers 200 can contain activated carbon. In some embodiments, a second replaceable container of the plurality of replaceable containers 200 can contain zirconium phosphate. In some embodiments, a third replaceable container of the plurality of replaceable containers 200 can contain zirconium phosphate. In some embodiments, a fourth replaceable container of the plurality of replaceable containers 200 can contain zirconium phosphate. In some embodiments, a fifth replaceable container of the plurality of replaceable containers 200 can contain zirconium oxide.

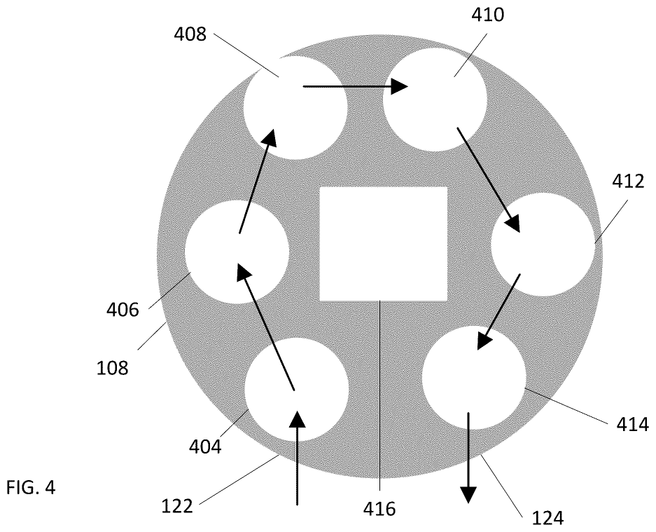

Turning to FIG. 4, the plurality of replaceable containers 200 can be organized, in series, based on the needs of the patient. The used dialysate can be conveyed along the dialysate pathway 120 into the dialysate fluid inlet 122. The dialysate can be transferred through each of the connected plurality of replaceable containers 200, in series, to filter out the waste products within the dialysate such that refreshed dialysate exits the sorbent cartridge 108 at the dialysate fluid outlet 124.

The sorbent cartridge 108 can include a plurality of sorbent materials arranged in series between the dialysate fluid inlet 122 and the dialysate fluid outlet 124. In the illustrated embodiment, the sorbent cartridge 108 includes a single dialysate fluid inlet 122 and a single dialysate fluid outlet 124. It is to be appreciated that this is an example and the actual number of dialysate fluid inlet 122 and the dialysate fluid outlet 124 can vary (e.g., multiple inlets, multiple outlets, or multiple inlets and multiple outlets). The sorbent materials can filter the waste products within the dialysate from the dialyzer 106. The plurality of sorbent materials can include at least three different kinds of materials as follows: 1) a zirconium phosphate (ZrP) material that has the capacity to act as a cation exchanger by absorbing a large quantity of ammonium ions in exchange for sodium and hydrogen ions; 2) a zirconium oxide material (ZrO), which acts as an anion exchanger by exchanging phosphate for acetate; and 3) an activated carbon material that has a surface area for adsorption of wide range of impurities including metal ions and uremic toxins, such as uric acid, creatinine, and ˜2-microglobin. The plurality of sorbent materials can contain a dissolvable element to improve their function or efficiency. For example, the dissolvable element can include a dissolvable foam that is configured to dissolve under presence of a fluid. In some embodiments, the foam is a water soluble foam. For example, the sorbent materials can include a dissolvable foam to address compression and/or hydration issues. In some embodiments, the dissolvable element can be used to prevent or eliminate particle segregation of the sorbent materials. In some embodiments, the dissolvable element can control a material bed within the sorbent material prior to application of a fluid. The dissolution of the element upon contact with a fluid can allow for the material bed within the sorbent material to expand without channeling or clumping upon introduction of the fluid. This can, for example, be more uniform as a result than with no dissolvable element present.

In the example shown in FIG. 4, the dialysate can enter the sorbent cartridge 108 through the dialysate fluid inlet 122 at which point it will go into a first container 404. In some embodiments, the first container 404 can include a urease-containing material. In some embodiments, the first container 404 can include activated carbon. The activated carbon can have a surface area for adsorption of wide range of impurities including metal ions and uremic toxins, such as uric acid, creatinine, and ˜2-microglobin. Once the dialysate travels through the first container 404, the dialysate can be conveyed, piped, or travel through a tube to a second container 406. The second container 406 can include ZrP to act as a cation exchanger by absorbing a large quantity of ammonium ions in exchange for sodium and hydrogen ions. There can be multiple of the plurality of replaceable containers 200 that contain the same filtering material. For example, a third container 408, a fourth container 410, and a fifth container 412 can also include ZrP. The fifth container 412 can act as a spare ZrP container which can be selectively used. In some embodiments, the fifth container 412 can be a redundant ZrP container. As the dialysate travels through the third container 408, the fourth container 410, and the fifth container 412, the dialysate can then flow into a sixth container 414. The sixth container 414 can include ZrO which can act as an anion exchanger by exchanging phosphate for acetate. Although discussed as six containers, it should be understood that this is merely an example, and the actual number of containers can vary according to the principles of this disclosure. For example, there may be more than 6 containers or there may be less than 6 containers. Moreover, the materials within each of the cartridges may vary, based on patient 102 needs.

The sorbent cartridge 108 can also include a recharge chamber 416 which can be selectively connected to at least one of the plurality of replaceable containers 200. The recharge chamber 416 can be selectively fluidly connected, for example, to the containers having ZrP, ZrO, or combinations thereof. In some embodiments, the recharge chamber 416 can be used to treat used or spent sorbent material so as to put the sorbent material back in condition for use. Upon a sorbent material undergoing “recharging,” the sorbent material can then be said to be “recharged.”

As dialysate travels through the plurality of replaceable containers 200, the filter materials within each of the plurality of replaceable containers 200 can require a recharge or refill of filtering materials so that the plurality of replaceable containers 200 can filter the waste products adequately. The recharge chamber 416 can include recharge solutions for the plurality of replaceable containers 200 so that the ZrP terminals, the ZrO terminals, or combinations thereof, can be recharged without physically removing said terminals. In some embodiments, the appropriate recharge solution can be selected so that upon passing the recharge solution through a particular one of the plurality of replaceable containers 200, the particular one of the plurality of replaceable containers 200 is in condition to be used to filter the used dialysate and produce the refreshed dialysate.

In some embodiments, recharging the plurality of replaceable containers 200 within the dialysis system 100 can remove the need for a recharge station ancillary to the dialysis system 100, which can take up more space in the house of the patient 102. In some embodiments, the recharge chamber 416 can include one or more salts and be filled by tap water at a location of the dialysis system 100. In some embodiments, the sorbent cartridge 108 can include a filter to filter the tap water for use in the dialysis system 100 or a separate filter can be included between the water source and the sorbent cartridge 108. As the tap water flows into the recharge chamber 416, the recharge solution can flow from the recharge chamber 416 and into one or more of the plurality of replaceable containers 200.

A method of use for the sorbent cartridge 108 is now described. An operator (e.g., the patient 102, a technician, or the like) can open a housing of the sorbent cartridge 108 to configure the sorbent materials therein for their specific needs. For example, the operator may install the first container 404 containing activated carbon, the second container 406 containing ZrP, the third container 408 containing ZrP, the fourth container 410 containing ZrP, the fifth container 412 containing ZrP, and the sixth container 414 containing ZrO, as recommended by a physician or the like. The operator can load (e.g., snap into place or the like) each of the plurality of replaceable containers 200 in the order described. Subsequently, the patient 102 initiates a dialysis treatment using the dialysis system 100. Used dialysate will begin filtering through the sorbent cartridge 108. In some embodiments, a counter can be used to trigger an indicator to the patient 102 that it is time to recharge one or more of the containers. For example, after N uses, an alert may be generated to trigger the recharging process. In some embodiments, the recharging process can be automatically triggered after the N uses. The operator can fill the recharge chamber 416 to a predetermined threshold with water. The water can be filtered by the sorbent cartridge 108 for use within the dialysis system 100. Then, the operator may selectively recharge the plurality of replaceable containers 200 having ZrP and ZrO and needing to be recharged using recharge solution from the recharge chamber 416. The dialysis system 100 can be ready for a next dialysis therapy.

The terminology used herein is intended to describe embodiments and is not intended to be limiting. The terms “a,” “an,” and “the” include the plural forms as well, unless clearly indicated otherwise. The terms “comprises” and/or “comprising,” when used in this Specification, specify the presence of the stated features, integers, steps, operations, elements, and/or components, but do not preclude the presence or addition of one or more other features, integers, steps, operations, elements, and/or components.

It is to be understood that changes may be made in detail, especially in matters of the construction materials employed and the shape, size, and arrangement of parts without departing from the scope of the present disclosure. This Specification and the embodiments described are examples, with the true scope and spirit of the disclosure being indicated by the claims that follow.

1. A sorbent cartridge comprising:
a fluid inlet;
a fluid outlet;
a plurality of sorbent materials arranged in series between the fluid inlet and the fluid outlet,
wherein each of the plurality of sorbent materials is configured to be disposed in a respective replaceable container;
wherein the plurality of sorbent materials are configured for fluid flow in a longitudinal direction of the respective replaceable container; and
a recharging solution disposed in a container,
wherein the recharging solution is configured to be selectively fluidly connected to one or more of the plurality of sorbent materials.

2. The sorbent cartridge of claim 1, wherein the respective replaceable container has a length and a diameter;
wherein the respective replaceable container has an aspect ratio defined by a ratio of the length to the diameter (length:diameter);
wherein the aspect ratio is at least 1.5.

3. The sorbent cartridge of claim 2, wherein the aspect ratio is at least 3.

4. The sorbent cartridge of claim 1, wherein one or more of the plurality of sorbent materials is in a form of a sorbent block.

5. The sorbent cartridge of claim 1, wherein the sorbent cartridge has a cylindrical geometry;
wherein the plurality of sorbent materials are arranged circumferentially around the sorbent cartridge in an order of flow.

6. The sorbent cartridge of claim 1, further comprising a dissolvable element within one or more of the plurality of sorbent materials.

7. The sorbent cartridge of claim 6, wherein the dissolvable element is configured to prevent particle segregation of a respective sorbent material in the respective replaceable container.

8. The sorbent cartridge of claim 1, wherein the respective replaceable container is configured to be installed within the sorbent cartridge using a snap in loader.

9. The sorbent cartridge of claim 1, wherein the plurality of sorbent materials are configured for fluid flow in a second direction other than the longitudinal direction.

10. The sorbent cartridge of claim 9, wherein the second direction includes a radial direction, a circumferential direction, or combination thereof.

11. A system comprising:
a dialysis system configured to output a fresh dialysate to a patient and configured to receive a used dialysate from the patient; and
a sorbent cartridge in the dialysis system and configured to receive the used dialysate and filter to generate a refreshed dialysate, the sorbent cartridge comprising:
a fluid inlet configured to receive the used dialysate;
a fluid outlet configured to output the refreshed dialysate;
a plurality of sorbent materials arranged in series between the fluid inlet and the fluid outlet,
wherein each of the plurality of sorbent materials is configured to be disposed in a respective replaceable container;
wherein the plurality of sorbent materials are configured for fluid flow in a longitudinal direction of the respective replaceable container; and
a recharging solution disposed in a container,
wherein the recharging solution is configured to be selectively fluidly connected to one or more of the plurality of sorbent materials.

12. The system of claim 11, wherein the respective replaceable container has a length and a diameter;
wherein the respective replaceable container has an aspect ratio defined by a ratio of the length to the diameter (length:diameter);
wherein the aspect ratio is at least 1.5.

13. The system of claim 11, wherein the sorbent cartridge has a cylindrical geometry;
wherein the plurality of sorbent materials are arranged circumferentially around the sorbent cartridge in an order of flow.

14. The system of claim 11, further comprising a dissolvable element within one or more of the plurality of sorbent materials.

15. The system of claim 14, wherein the dissolvable element is configured to prevent particle segregation of a respective sorbent material in the respective replaceable container.

16. The system of claim 11, wherein the plurality of sorbent materials are configured for fluid flow in a second direction other than the longitudinal direction.

17. A device comprising:
a housing comprising a fluid inlet and a fluid outlet;
a plurality of terminals configured to be fluidly connected to a connector of a respective sorbent material,
wherein the plurality of terminals are fluidly connected so that the respective sorbent material is fluidly connected when the respective sorbent material is installed in the plurality of terminals; and
a recharging solution container configured to be selectively fluidly connected to one or more of the plurality of terminals.

18. The device of claim 17, wherein the housing is cylindrical and the plurality of terminals are arranged about a circumference of the housing.

19. The device of claim 17, wherein the respective sorbent material comprises one or more of activated carbon, zirconium phosphate, or zirconium oxide.

20. The device of claim 19, wherein a first of the plurality of terminals is configured to receive the zirconium phosphate or the zirconium oxide; wherein the recharging solution container is configured to be selectively fluidly connected to the first of the plurality of terminals.
